(19) United States
(12) Patent Application Publication
(10) Pub. No.: US 2021/0166803 A1
(43) Pub. Date: Jun. 3, 2021

(54) RECOMMENDING ACTIONS FOR AVOIDANCE OF FOOD INTOLERANCE SYMPTOMS

(71) Applicant: International Business Machines Corporation, Armonk, NY (US)

(72) Inventors: Laura Grace Ellis, Austin, TX (US); Shikhar Kwatra, Durham, NC (US); Corinne Anne Leopold, Austin, TX (US); Sarbajit K. Rakshit, Kolkata (IN)

(21) Appl. No.: 16/699,979

(22) Filed: Dec. 2, 2019

(51) Int. Cl. G16H 20/60; G06N 3/04; G06F 3/01; H04W 4/029; G06K 9/00

(52) U.S. Cl. CPC G16H 20/60; G06N 3/0454; G06K 9/00671; H04W 4/029; G06F 3/011

(57) ABSTRACT

In an approach to recommending actions for avoiding food intolerance, one or more computer processors receive data, where the received data includes data associated with a food item viewed by a first user, data associated with the first user, and data associated with an environment of the first user. One or more computer processors determine a health condition of the first user. One or more computer processors predict a first food intolerance reaction of the first user to the viewed food item based on the received data and the determined health condition. One or more computer processors determine a first action recommendation for the first user corresponding to the first predicted food intolerance reaction. One or more computer processors determine a first action recommendation for the first user corresponding to the first predicted food intolerance reaction. One or more computer processors present the first action recommendation to the first user.

### BACKGROUND OF THE INVENTION

The present invention relates generally to the field of machine learning, and more particularly to a reinforcement learning model for recommending actions for avoiding food intolerance symptoms.

Reinforcement learning is an area of machine learning in which a model or algorithm is trained to take a suitable action to maximize reward in a particular situation. Reinforcement learning is employed by various software and machines to find the best possible behavior or path to take in a specific situation. Reinforcement learning differs from supervised learning. In supervised learning, the training data includes the correct answers. In reinforcement learning, there is no answer, but the reinforcement agent decides what to do to perform the given task. In the absence of training dataset, the model is bound to learn from experience. Reinforcement learning involves making decisions sequentially. In other words, the output depends on the state of the current input and the next input depends on the output of the previous input.

The internet of things (IoT) is the internetworking of physical devices (also referred to as “connected devices” and “smart devices”), vehicles, buildings, and other items, embedded with electronics, software, sensors, actuators, and network connectivity that enable these objects to collect and exchange data. The IoT allows objects to be sensed and/or controlled remotely across existing network infrastructure, creating opportunities for more direct integration of the physical world into computer-based systems, and resulting in improved efficiency, accuracy, and economic benefit in addition to reduced human intervention. Each thing is uniquely identifiable through its embedded computing system but is able to interoperate within the existing Internet infrastructure.

Food intolerance is a detrimental reaction, often delayed, to a food, beverage, food additive, or compound found in foods that produces symptoms in one or more body organs and systems, but generally refers to a difficulty in digesting certain foods. Foods most commonly associated with food intolerance include dairy products, grains that contain gluten, and foods that cause intestinal distress. Food intolerance reactions can include pharmacologic, metabolic, and gastro-intestinal responses to foods or food compounds. Symptoms of food intolerance may include, but are not limited to, digestive ailments, migraines, headaches, cough, runny nose, feeling under the weather, and hives.

### SUMMARY

Embodiments of the present invention disclose a method, a computer program product, and a system for recommending actions for avoiding food intolerance symptoms. The method may include one or more computer processors receiving data, where the received data includes data associated with a food item viewed by a first user, data associated with the first user, and data associated with an environment of the first user. One or more computer processors determine a first health condition of the first user. One or more computer processors predict a first food intolerance reaction of the first user to the viewed food item based on the received data and the determined first health condition. One or more computer processors determine a first action recommendation for the first user corresponding to the first predicted food intolerance reaction. One or more computer processors determine a first action recommendation for the first user corresponding to the first predicted food intolerance reaction. One or more computer processors present the first action recommendation to the first user.

### BRIEF DESCRIPTION OF THE DRAWINGS

FIG. 1 is a functional block diagram illustrating a distributed data processing environment, in accordance with an embodiment of the present invention;

FIG. 2 is a flowchart depicting operational steps of a food intolerance system, on a server computer within the distributed data processing environment of FIG. 1, for training a food intolerance model, in accordance with an embodiment of the present invention;

FIG. 3 is a flowchart depicting operational steps of the food intolerance system, on the server computer within the distributed data processing environment of FIG. 1, for making recommendations regarding food intake, in accordance with an embodiment of the present invention; and

FIG. 4 depicts a block diagram of components of the server computer executing the food intolerance system within the distributed data processing environment of FIG. 1, in accordance with an embodiment of the present invention.

### DETAILED DESCRIPTION

A food intolerance, or a food sensitivity, occurs when a person's digestive system cannot tolerate certain foods and reacts with one or more physical symptoms as a result of the person consuming the food, thereby possibly restricting a person's food selections. In addition to intolerance of the food itself, a person's food intolerance may be affected by a current health condition, activities performed before or after consuming the food, weather conditions, and compatibility of food combinations, i.e., eating certain foods together or in a particular sequence.

Embodiments of the present invention recognize that providing a person with a recommendation to eat or not eat a selected food based on a knowledge corpus can prevent illness due to food intolerance. Embodiments of the present invention also recognize that efficiency may be gained by receiving data corresponding to a food selection via one or more sensors operably coupled to a user and/or operably coupled to one or more Internet of Things (IoT) devices to feed into a reinforcement learning model for predicting food intolerance and displaying an action recommendation to the user. Implementation of embodiments of the invention may take a variety of forms, and exemplary implementation details are discussed subsequently with reference to the Figures.

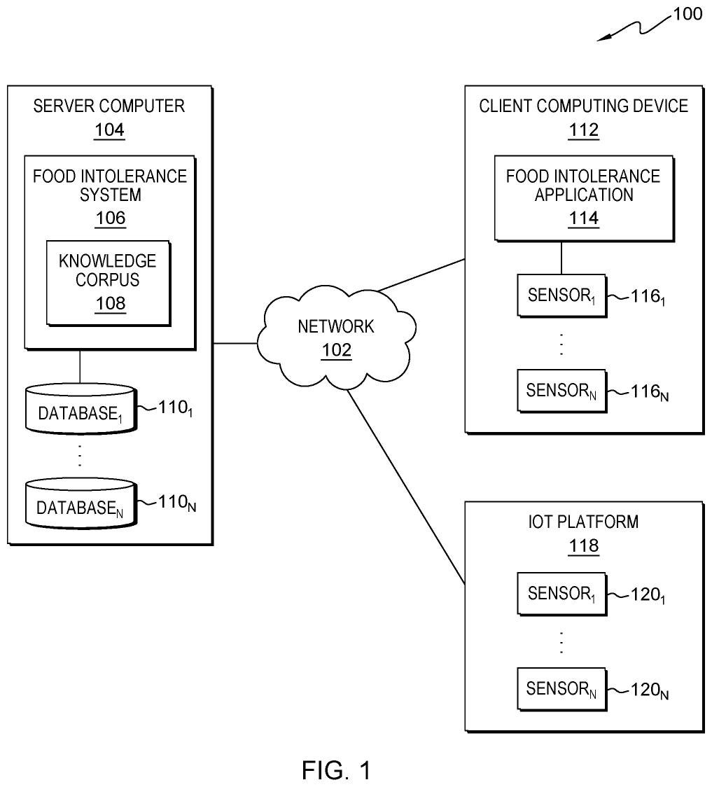

FIG. 1 is a functional block diagram illustrating a distributed data processing environment, generally designated 100, in accordance with one embodiment of the present invention. The term “distributed” as used herein describes a computer system that includes multiple, physically distinct devices that operate together as a single computer system. FIG. 1 provides only an illustration of one implementation and does not imply any limitations with regard to the environments in which different embodiments may be implemented. Many modifications to the depicted environment may be made by those skilled in the art without departing from the scope of the invention as recited by the claims.

Distributed data processing environment 100 includes server computer 104, client computing device 112, and internet of things (IoT) platform 118, all interconnected over network 102. Network 102 can be, for example, a telecommunications network, a local area network (LAN), a wide area network (WAN), such as the Internet, or a combination of the three, and can include wired, wireless, or fiber optic connections. Network 102 can include one or more wired and/or wireless networks capable of receiving and transmitting data, voice, and/or video signals, including multimedia signals that include voice, data, and video information. In general, network 102 can be any combination of connections and protocols that will support communications between server computer 104, client computing device 112, IoT platform 118, and other computing devices (not shown) within distributed data processing environment 100.

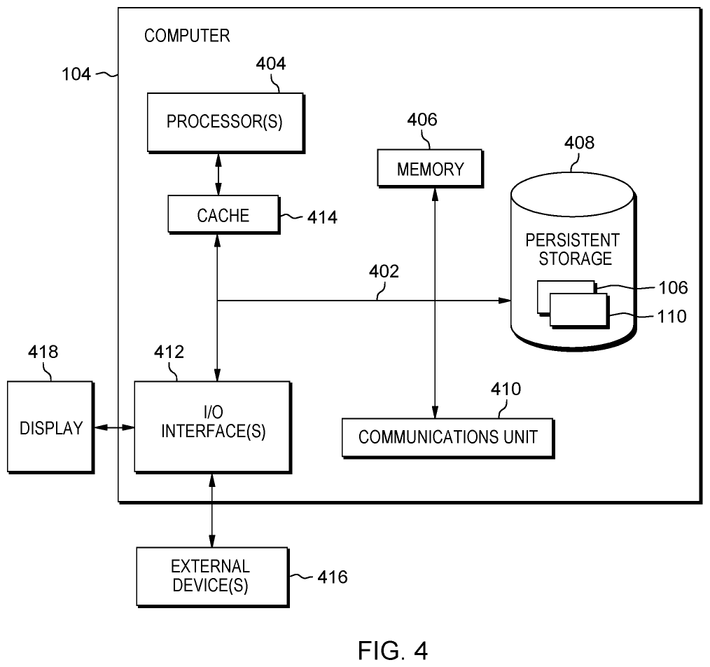

Server computer 104 can be a standalone computing device, a management server, a web server, a mobile computing device, or any other electronic device or computing system capable of receiving, sending, and processing data. In other embodiments, server computer 104 can represent a server computing system utilizing multiple computers as a server system, such as in a cloud computing environment. In another embodiment, server computer 104 can be a laptop computer, a tablet computer, a netbook computer, a personal computer (PC), a desktop computer, a personal digital assistant (PDA), a smart phone, or any programmable electronic device capable of communicating with client computing device 112, IoT platform 118, and other computing devices (not shown) within distributed data processing environment 100 via network 102. In another embodiment, server computer 104 represents a computing system utilizing clustered computers and components (e.g., database server computers, application server computers, etc.) that act as a single pool of seamless resources when accessed within distributed data processing environment 100. Server computer 104 includes food intolerance system 106 and database 110_{1-N}. Server computer 104 may include internal and external hardware components, as depicted and described in further detail with respect to FIG. 4.

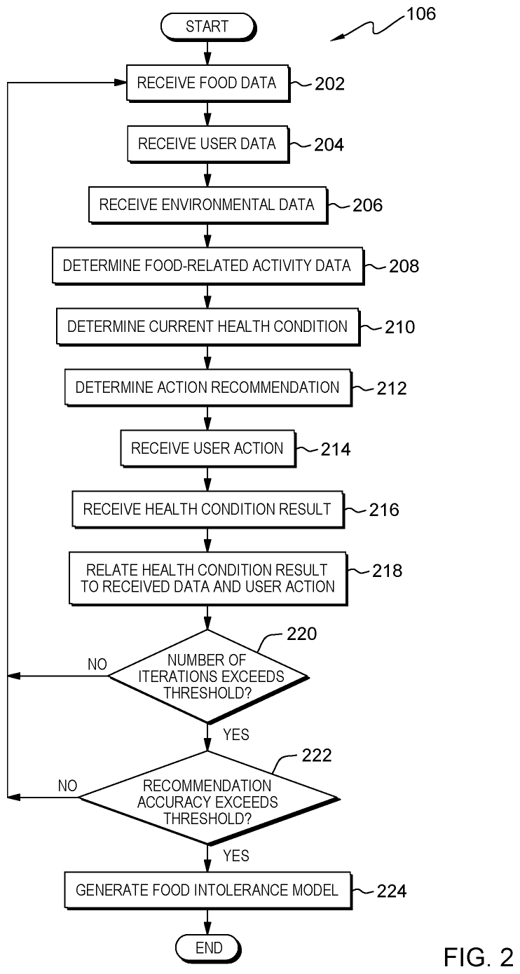

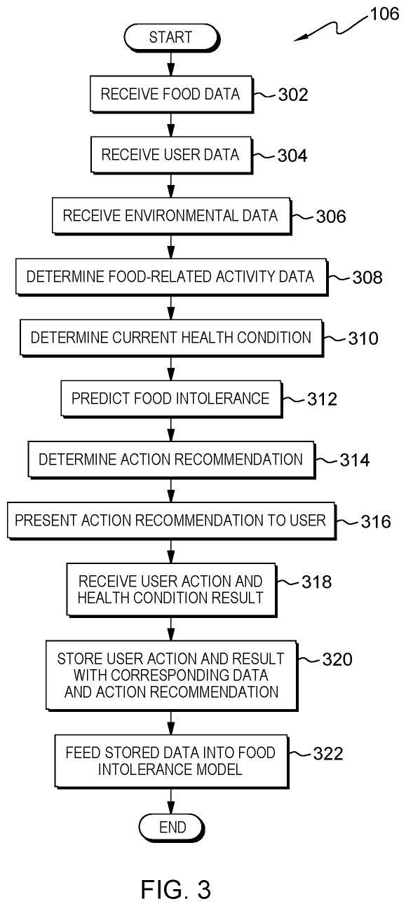

Food intolerance system 106 uses a reinforcement learning model (not shown), referred to herein as a food intolerance model, to determine one or more actions for a user to take in order to avoid symptoms of food intolerance when the user considers making a food selection. During a training phase, food intolerance system 106 receives data regarding a potential food selection by a user. Food intolerance system 106 also receives data specific to a user and any activities performed, or planned to be performed, by the user which may relate to the food selection. Food intolerance system 106 also receives data corresponding to the environment of the user. Food intolerance system 106 determines a health condition of the user. Based on the received data and the health condition of the user, food intolerance system 106 determines an action recommendation for the user with respect to the food selection. Food intolerance system 106 receives a user action and a resulting health condition. Food intolerance system 106 relates the health condition to the corresponding received data and user action. Food intolerance system 106 repeats the above-mentioned actions for a threshold number of iterations, at which time food intolerance system 106 determines an accuracy of the recommendations. If the accuracy of the recommendations, i.e., whether the recommendations would have resulted in a positive outcome for the user, exceeds a threshold, then food intolerance system 106 generates a food intolerance model. If the accuracy of the recommendations does not exceed the threshold, then food intolerance system 106 continues to process data, recommendations, and actions until the accuracy is at an acceptable level. Once trained, food intolerance system 106 receives data regarding a potential food selection by a user, along with associated user data, environmental data, and data specific to any activities performed, or planned to be performed, by the user which may relate to the food selection. Based on the received data, and using the food intolerance model, food intolerance system 106 predicts a food intolerance by the user associated with the potential food selection. Food intolerance system 106 determines a recommended action for the user and displays the recommendation to the user. Food intolerance system 106 receives a user action and a health condition result of the user action. Food intolerance system 106 stores the user action and health condition result of the user action with the corresponding user data, environmental data, and food-related activity, and feeds the stored data into the food intolerance model in order to continue to train the model for increasing accuracy of recommendations. Food intolerance system 106 includes knowledge corpus 108. Food intolerance system 106 is depicted and described in further detail with respect to FIG. 2 and FIG. 3.

Knowledge corpus 108 is a data repository for food intolerance system 106. In the depicted embodiment, knowledge corpus 108 is integrated within food intolerance system 106. In another embodiment, knowledge corpus 108 may reside elsewhere on server computer 104 or elsewhere within distributed data processing environment 100, provided food intolerance system 106 has access to knowledge corpus 108. In an embodiment, knowledge corpus 108 can be implemented with any type of storage device capable of storing data and configuration files that can be accessed and utilized by food intolerance system 106, such as a database server, a hard disk drive, or a flash memory. Knowledge corpus 108 stores data received or retrieved by food intolerance system 106 for determining a food-related action recommendation for a user. Knowledge corpus 108 also stores the recommendations and the user actions in response to the recommendations, and the results of the user actions. In addition, knowledge corpus 108 stores the relationship determined by food intolerance system 106 between the received/retrieved data, the recommendations, the user actions, and the result of the actions.

Database 110_{1-N}, herein database(s) 110, are each a repository for data used by food intolerance system 106. As used herein, N represents a positive integer, and accordingly the number of scenarios implemented in a given embodiment of the present invention is not limited to those depicted in FIG. 1. Database(s) 110 may each represent one or more databases. In the depicted embodiment, database(s) 110 reside on server computer 104. In another embodiment, database(s) 110 may each reside elsewhere within distributed data processing environment 100 provided food intolerance system 106 has access to database(s) 110. A database is an organized collection of data. Database(s) 110 can each be implemented with any type of storage device capable of storing data and configuration files that can be accessed and utilized by food intolerance system 106, such as a database server, a hard disk drive, or a flash memory. Database(s) 110 store food data retrieved by food intolerance system 106 in an effort to identify a user's food selection and the associated properties and ingredients.

Client computing device 112 can be one or more of a laptop computer, a tablet computer, a smart phone, smart watch, a smart speaker, or any programmable electronic device capable of communicating with various components and devices within distributed data processing environment 100, via network 102. Client computing device 112 may be a wearable computer. Wearable computers are miniature electronic devices that may be worn by the bearer under, with, or on top of clothing, as well as in or connected to glasses, hats, or other accessories. Wearable computers are especially useful for applications that require more complex computational support than merely hardware coded logics. In one embodiment, the wearable computer may be in the form of a head mounted display. The head mounted display may take the form-factor of a pair of glasses. In an embodiment, the glasses are capable of displaying augmented reality objects in the field of view of the user. In an embodiment, the wearable computer may be in the form of a smart watch. In an embodiment, client computing device 112 may be integrated into a vehicle of the user. In general, client computing device 112 represents one or more programmable electronic devices or combination of programmable electronic devices capable of executing machine readable program instructions and communicating with other computing devices (not shown) within distributed data processing environment 100 via a network, such as network 102. Client computing device 112 includes an instance of food intolerance application 114 and sensor 116_{1-N}.

Food intolerance application 114 provides an interface between food intolerance system 106 on server computer 104 and a user of client computing device 112. In one embodiment, food intolerance application 114 is mobile application software. Mobile application software, or an “app,” is a computer program designed to run on smart phones, tablet computers and other mobile devices. In one embodiment, food intolerance application 114 may be a graphical user interface (GUI) or a web user interface (WUI) and can display text, documents, web browser windows, user options, application interfaces, and instructions for operation, and include the information (such as graphic, text, and sound) that a program presents to a user and the control sequences the user employs to control the program. Food intolerance application 114 enables a user of client computing device 112 to provide preferences and health information to food intolerance system 106 to continually train food intolerance system 106 to recommend food intolerance related actions. Food intolerance application 114 also enables the user of client computing device 112 to receive food intolerance related recommendations from food intolerance system 106.

Internet of things (IoT) platform 118 is a suite of components that enable a) deployment of applications that monitor, manage, and control connected devices and sensors; b) remote data collection from connected devices; and c) independent and secure connectivity between devices. The components may include, but are not limited to, a hardware architecture, an operating system, or a runtime library (not shown). In the depicted embodiment, IoT platform 118 includes sensor 120_{1-N}. In another embodiment, IoT platform 118 may include a plurality of other connected computing devices. For example, IoT platform 118 may include home security devices, electronic assistants, etc. In another example, IoT platform 118 may include a home climate control system or various kitchen appliances. In a further example, IoT platform 118 may be a restaurant or grocery store information system.

A sensor is a device that detects or measures a physical property and then records or otherwise responds to that property, such as vibration, chemicals, radio frequencies, environment, weather, humidity, light, etc. Sensor 116_{1-N} and sensor 120_{1-N}, herein sensor(s) 116 and sensor(s) 120, detect a plurality of attributes of a user of food intolerance application 114 and of food and food venues in a plurality of locations. As used herein, N represents a positive integer, and accordingly the number of scenarios implemented in a given embodiment of the present invention is not limited to those depicted in FIG. 1. Sensor(s) 116 and sensor(s) 120 may be one or more of a plurality of types of camera, including, but not limited to, pin-hole, stereo, omni-directional, non-central, infrared, video, digital, three dimensional, panoramic, filter-based, wide-field, narrow-field, telescopic, microscopic, etc. In some embodiments, sensor(s) 116 and sensor(s) 120 include any device capable of imaging a portion of the electromagnetic spectrum. If client computing device 112 is a wearable device, then sensor(s) 116 may include biometric sensors for detecting the physical condition of the user, such as blood pressure, heart rate, respiratory rate, calories burned, calories consumed, pulse, oxygen levels, blood oxygen level, glucose level, blood pH level, salinity of user perspiration, skin temperature, galvanic skin response, electrocardiography (ECG or EKG) data, body temperature, eye tracking data, etc. A biometric sensor may also be an ingestible gut sensor for detecting issues in the user's digestive tract. Sensor(s) 116 and sensor(s) 120 may be one or more of a plurality of types of microphone for detecting speech and other audible sounds. Sensor(s) 116 and sensor(s) 120 may be able to detect weather conditions, such as air temperature, relative humidity, presence and type of precipitation, wind speed, etc., as food intolerance may depend on the weather conditions. Sensor(s) 116 and sensor(s) 120 may be global positioning system (GPS) sensors or other sensors that can determine a location of client computing device 112. Sensor(s) 116 and sensor(s) 120 may also track a user's mobility pattern, for example, whether the user lies down after eating a particular food. Sensor(s) 116 may be integrated into the vehicle of the user.

The present invention may contain various accessible data sources, such as database(s) 110, that may include personal data, content, or information the user wishes not to be processed. Personal data includes personally identifying information or sensitive personal information as well as user information, such as tracking or geolocation information. Processing refers to any, automated or unautomated, operation or set of operations such as collection, recording, organization, structuring, storage, adaptation, alteration, retrieval, consultation, use, disclosure by transmission, dissemination, or otherwise making available, combination, restriction, erasure, or destruction performed on personal data. Food intolerance system 106 and food intolerance application 114 enable the authorized and secure processing of personal data. Food intolerance system 106 and food intolerance application 114 provide informed consent, with notice of the collection of personal data, allowing the user to opt in or opt out of processing personal data. Consent can take several forms. Opt-in consent can impose on the user to take an affirmative action before personal data is processed. Alternatively, opt-out consent can impose on the user to take an affirmative action to prevent the processing of personal data before personal data is processed. Food intolerance system 106 and food intolerance application 114 provide information regarding personal data and the nature (e.g., type, scope, purpose, duration, etc.) of the processing. Food intolerance system 106 and food intolerance application 114 provide the user with copies of stored personal data. Food intolerance system 106 and food intolerance application 114 allow the correction or completion of incorrect or incomplete personal data. Food intolerance system 106 and food intolerance application 114 allow the immediate deletion of personal data.

FIG. 2 is a flowchart depicting operational steps of food intolerance system 106, on server computer 104 within distributed data processing environment 100 of FIG. 1, for training a food intolerance model, in accordance with an embodiment of the present invention.

Food intolerance system 106 receives food data (step 202). In an embodiment, as a user views and considers selecting a food, for example scanning a menu in a restaurant, looking at a buffet table, viewing shelves in a grocery store, etc., food intolerance system 106 receives an image of the selected food via food intolerance application 114. In another embodiment, food intolerance system 106 may receive food data by scanning the text of a food menu and analyzing the text. Food intolerance system 106 receives food data by matching the image or text associated with the selected food with the food identification and data associated with the food in one or more of database(s) 110. Data associated with a food selection includes, but is not limited to, the type of food, the quantity of food chosen, the ingredients of the food, one or more properties of the food, frequency of consumption of the food, the quality of the food, one or more certifications associated with the food, such as whether the food is “certified organic,” etc. In an embodiment, food data can also include the time, sequence, and/or frequency of other food items consumed by the user within a threshold duration of time in order to enable food intolerance system 106 to observe any correlation of a food intolerance to an interaction between different foods. For example, a user may avoid discomfort from consuming a cup of coffee if the user consumes a glass of water first. In an embodiment in which client computing device 112 is a wearable computer in the form of glasses operably coupled to a camera, food intolerance system 106 receives food data by capturing one or more images of the food selection within the gaze of the user and retrieving any data stored in database(s) 110 associated with the food selection. In the embodiment, if the user is viewing items in a grocery store, food intolerance system 106 may receive food data by instructing client computing device 112 to scan a QR code or a bar code on the food package or on the shelf In an embodiment in which client computing device 112 is a smart phone, food intolerance system 106 may instruct the user to capture one or more images of the food and food packaging, as well as scanning a QR code or a bar code on the food package or on the shelf with client computing device 112 and retrieving any data stored in database(s) 110 associated with the food selection. In another embodiment, food intolerance system 106 may receive images of the food selection from one or more of sensor(s) 120. In an embodiment, food intolerance system 106 stores the received food data in knowledge corpus 108. In an embodiment where sensor(s) 116 can track hand movements or gestures of the user, food intolerance system 106 may initiate food data gathering based on receiving hand movements or gestures that indicate the user is about to select a food item.

Food intolerance system 106 receives user data (step 204). In an embodiment, food intolerance system 106 receives user data via food intolerance application 114. In an embodiment, a user completes a user profile in food intolerance application 114. The user data entered in the user profile may include physical data about a user, for example, the age, height, and weight of a user, and a medical history of a user which may include medical test results. In an embodiment, a user may enter specifics about food intolerances and/or food allergies into food intolerance application 114 to be included in user data. Additional user data may include other dietary restrictions, such as due to a health concern. In addition, user data may include food likes and dislikes of a user. In an embodiment, food intolerance system 106 may receive user data via crowdsourcing from, for example, one or more social networks. For example, food intolerance system 106 may determine that a number of users were ill after eating at a particular restaurant. In an embodiment, food intolerance system 106 stores the received user data in knowledge corpus 108.

Food intolerance system 106 receives environmental data (step 206). In an embodiment, food intolerance system 106 receives environmental data from sensor(s) 116 or sensor(s) 120, or both. For example, food intolerance system 106 may receive current weather conditions, including, but not limited to, a temperature, a wind speed, a presence and type of precipitation, a sky condition, such as whether it is sunny or cloudy, etc. Food intolerance system 106 may also receive data indicating the current location of client computing device 112. For example, if one or more of sensor(s) 116 include a GPS sensor, then food intolerance system 106 may determine the physical location of client computing device 112. In another example, food intolerance system 106 may determine whether the user is in a grocery store or in a restaurant by analyzing images received from sensor(s) 116 and/or sensor(s) 120. In an embodiment, food intolerance system 106 stores the received environmental data in knowledge corpus 108.

Food intolerance system 106 determines food-related activity data (step 208). The sequence of activities a user participates in before or after consuming a particular food can impact a food intolerance response. Food intolerance system 106 determines one or more activities of the user that may correspond to or impact a food intolerance response. In an embodiment, food intolerance system 106 retrieves data from an electronic calendar on client computing device 112 (not shown) which indicates one or more activities the user participated in prior to the food selection or activities the user plans to participate in following the food selection. For example, food intolerance system 106 may determine that the user just completed a work day where the user was in several meetings, and therefore sedentary for most of the day. In another example, food intolerance system 106 may determine that after the food selection activity, the user is going to participate in a vigorous physical activity. In another embodiment, food intolerance system 106 may determine a prior physical activity by receiving data from one or more of sensor(s) 116. For example, food intolerance system 106 may detect an elevated heart rate or perspiration which indicates the user just completed a physical activity. In a further embodiment, food intolerance system 106 may determine a prior or future activity of the user by receiving one or more images of the user from one or more or sensor(s) 116 and/or sensor(s) 120. For example, food intolerance system 106 may receive an image which depicts the clothing the user is wearing which may indicate a prior or future activity. In an embodiment, the food-related activity may also be a meal or snack the user had previously. In an embodiment, the food-related activity may also be an adverse reaction to a food the user ingested prior to the current food selection. In yet another embodiment, food intolerance system 106 may determine a food-related activity by retrieving data associated with the user from one or more social networks. In an embodiment, food intolerance system 106 stores the determined food-related activity data in knowledge corpus 108.

Food intolerance system 106 determines current health condition (step 210). In an embodiment, food intolerance system 106 may receive user data from one or more of sensor(s) 116 that indicates the current health conditions of the user. For example, food intolerance system 106 may receive biometric data, including, but not limited to, blood pressure, heart rate, respiratory rate, calories burned, calories consumed, pulse, oxygen levels, blood oxygen level, glucose level, blood pH level, salinity of user perspiration, skin temperature, galvanic skin response, electrocardiography (ECG or EKG) data, body temperature, eye tracking data, mobility data, etc. In another embodiment, food intolerance system 106 may receive current health conditions from one or more of sensor(s) 120. For example, food intolerance system 106 may receive images of a user that depict the physical condition of the user, such as whether the user is sweating or shivering. In another example, food intolerance system 106 may receive images of a user that depict a change in the user's mobility, i.e., the user lies down. In an embodiment where one or more of sensor(s) 116 and/or sensor(s) 120 is a microphone, food intolerance system 106 may receive current health condition by detecting speech by the user and using one or more natural language processing (NLP) techniques to understand what the user is saying. For example, if the user states “I have a bad stomachache,” then food intolerance system 106 determines the user's current health condition from the speech analysis. In an embodiment, food intolerance system 106 stores the determined health condition data in knowledge corpus 108.

Food intolerance system 106 determines an action recommendation (step 212). Based on the received food data, user data, and environmental data, and on the one or more determined food-related activities and the determined current health condition of the user, food intolerance system 106 determines an action recommendation corresponding to the food item the user is viewing or has selected. In an embodiment, food intolerance system 106 retrieves the data from knowledge corpus 108. As data is accumulated and knowledge corpus 108 grows, food intolerance system 106 begins a process of reinforcement learning in order to determine an action to recommend to the user. In an embodiment, the recommended action is either to eat the selected food or to not eat the selected food. Over time, based on the available data, food intolerance system 106 determines which foods, or combinations of foods, or combinations of foods with food-related activities, or combinations of food with one or more weather conditions, can result in an adverse effect on the user. In an embodiment, food intolerance system 106 stores the determined action recommendations in knowledge corpus 108.

Food intolerance system 106 receives user action (step 214). Using data received from sensor(s) 116 and/or sensor(s) 120, food intolerance system 106 receives the user action regarding the food selection. For example, food intolerance system 106 may receive one or more images of the user placing the food in a shopping cart or on a plate. In another example, food intolerance system 106 may receive an image of the user putting the food back on the grocery store shelf. In a further example, food intolerance system 106 may receive an indication that the user ate the selected food. In an embodiment, food intolerance system 106 may receive the user action when the user inputs the action in food intolerance application 114. In an embodiment, food intolerance system 106 stores the received user action data in knowledge corpus 108.

Food intolerance system 106 receives health condition result (step 216). In an embodiment, food intolerance system 106 receives an updated health condition of the user in response to the user action. In one embodiment, food intolerance system 106 waits for a threshold duration of time to pass before receiving the health condition result in order to take into account a delayed response a food selection might have on a user's health. For example, food intolerance system 106 may receive the health condition result a half hour after the user action if the user action was to select food from a buffet and place the selected food on a plate. Food intolerance system 106 may receive the health condition result in a plurality of ways, as discussed with respect to step 210. If the user ate the selected food, then food intolerance system 106 determines whether the health of the user was positively or adversely affected. For example, food intolerance system 106 can determine whether the user grimaced and rubbed the user's stomach after eating the food. If the user did not eat the food, then food intolerance system 106 determines whether the health of the user was positively or adversely affected. For example, if the user appeared ill prior to the food selection and the user did not eat the selected food, then food intolerance system 106 can determine whether or not the user's health improved. In an embodiment, food intolerance system 106 stores the received health condition result in knowledge corpus 108. Food intolerance system 106 uses the determination of whether the user was positively or adversely affected by an action recommendation in the reinforcement learning process by “rewarding” a positive outcome and “penalizing” a negative outcome, enabling optimization for the reward function.

Food intolerance system 106 relates health condition result to received data and user action (step 218). In an embodiment, food intolerance system 106 creates or updates an existing matrix of food data, user data, environmental data, corresponding food-related activities, and the health condition of the user before and after the food selection, with the determined action recommendation and user action in an effort to predict the effect a recommended action has in response to a food selection. For example, food intolerance system 106 may determine how the user's health condition changes as a result of eating a particular food. In another example, food intolerance system 106 may determine how a change in health condition is related to a quantity of food intake. In a further example, food intolerance system 106 may determine how a change in health condition is related to a sequence of food intake, such as the user avoiding discomfort from consuming a cup of coffee when the user consumes a glass of water first. In yet another example, food intolerance system 106 may determine how a user's medical condition is related to a food intake-based illness. In another example, food intolerance system 106 may determine how weather is related to a food intake-based illness. In general, food intolerance system 106 determines which foods can create a health problem for a user. In an embodiment, food intolerance system 106 relates this data within knowledge corpus 108.

Food intolerance system 106 determines whether a number of iterations exceeds a threshold (decision block 220). In an embodiment, a system administrator pre-defines a quantity of iterations of steps 202 through 218 for food intolerance system 106 to complete in order to ensure that food intolerance system 106 has enough data to generate a food intolerance model with required accuracy.

If food intolerance system 106 determines the number of iterations does not exceed a threshold (“no” branch, decision block 220), then food intolerance system 106 returns to step 202 to increase the number of iterations through the food intolerance model training process.

If food intolerance system 106 determines the number of iterations exceeds a threshold (“yes” branch, decision block 220), then food intolerance system 106 determines whether the recommendation accuracy exceeds a threshold (decision block 222). Food intolerance system 106 analyzes the previously recommended actions from each iteration of the food intolerance model training process with respect to the actual actions taken by the user to determine whether the recommended actions would have produced a desired outcome for the user. In an embodiment, the measure of accuracy equals the number of actions that would have produced desirable results divided by the total number of determined action recommendations. In an embodiment, a system administrator pre-defines the threshold for accuracy. For example, the threshold may be ninety percent, such that if the accuracy is greater than ninety percent, then the accuracy exceeds the threshold.

If food intolerance system 106 determines the recommendation accuracy does not exceed a threshold (“no” branch, decision block 222), then food intolerance system 106 returns to step 202 to increase the number of iterations through the food intolerance model training process.

If food intolerance system 106 determines the recommendation accuracy exceeds a threshold (“yes” branch, decision block 222), then food intolerance system 106 generates a food intolerance model (step 224). Once the accuracy of the recommendations is sufficient, food intolerance system 106 generates a food intolerance model. Food intolerance system 106 feeds the gathered feature data, as described with respect to step 218, into the food intolerance model. In an embodiment, food intolerance system 106 bases the food intolerance model on the data in knowledge corpus 108. Initially, the food intolerance model is a baseline model because the data in knowledge corpus 108 is gathered from a variety of general users and from crowdsourcing. As will be discussed with respect to FIG. 3, as each additional specific user adopts the use of food intolerance application 114, food intolerance system 106 retrieves data specific to each user, and, through learning transfer, food intolerance system 106 creates and trains a specific version of the food intolerance model for each user by removing a general user classification and providing a user specific classification.

FIG. 3 is a flowchart depicting operational steps of food intolerance system 106, on server computer 104 within distributed data processing environment 100 of FIG. 1, for making recommendations regarding food intake, in accordance with an embodiment of the present invention.

Food intolerance system 106 receives food data (step 302). Once food intolerance system 106 completes initial training of the food intolerance model using a variety of users, a specific user can adopt food intolerance system 106 by using food intolerance application 114. Food intolerance system 106 receives food data from the specific user as discussed with respect to step 202 of FIG. 2.

Food intolerance system 106 receives user data (step 304). Food intolerance system 106 receives user data from the specific user as discussed with respect to step 204 of FIG. 2.

Food intolerance system 106 receives environmental data (step 306). Food intolerance system 106 receives environmental data associated with the specific user as discussed with respect to step 206 of FIG. 2.

Food intolerance system 106 receives food-related activity data (step 308). Food intolerance system 106 receives food-related activity data associated with the specific user as discussed with respect to step 208 of FIG. 2.

Food intolerance system 106 determines a current health condition (step 310). Food intolerance system 106 determines the current health condition of the specific user as discussed with respect to step 210 of FIG. 2.

Food intolerance system 106 predicts food intolerance (step 312). Food intolerance system 106 feeds the received food data, received user data, received environmental data, and received food-related activity data into the food intolerance model to predict whether the user may experience food intolerance symptoms as a result of eating the selected food. In an embodiment, food intolerance system 106 may predict the timing of the food intolerance symptoms. For example, food intolerance system 106 may determine that the user will begin experiencing food intolerance symptoms within an hour of consuming the food. In another embodiment, food intolerance system 106 may predict a relationship between the user consuming the selected food and the next activity the user plans to perform. For example, if food intolerance system 106 determines the food intolerance is exercise-dependent, then food intolerance system 106 may predict that the user will experience food intolerance symptoms if the user rides a bicycle within an hour of consuming the selected food.

Food intolerance system 106 determines an action recommendation (step 314). Based on the information in knowledge corpus 108, the data received in steps 302 through 308, and the predicted food intolerance, food intolerance system 106 determines an action recommendation. In one embodiment, the recommendation is either eat the food or do not eat the food. In another embodiment, the recommendation may include one or more caveats. For example, the recommendation may be do not eat the food until after exercise. In another example, the recommendation may be to eat the food before the temperature outside reaches 80 degrees Fahrenheit. In a further example, the recommendation may be to allow a time gap between the previous meal and consuming the selected food. In yet another example, the recommendation may be for a quantity of food to eat.

Food intolerance system 106 presents the action recommendation to the user (step 316). In an embodiment where client computing device 112 is a head mounted display in the form of glasses, food intolerance system 106 displays the action recommendation as augmented reality in the field of view of the user. For example, food intolerance system 106 may display text such as “Do not eat the meatloaf! Its ingredients include eggs.” In another example, food intolerance system 106 may overlay text or symbols over various foods in the view of the user which indicate which foods to eat and which foods to avoid, such as a circle around recommended foods to eat and an “X” over foods to avoid. In an embodiment where client computing device 112 is a smart phone, food intolerance system 106 displays the action recommendation on the screen of the smart phone. In an embodiment where client computing device 112 is a smart watch, food intolerance system 106 displays the action recommendation on the screen of the smart watch. In another embodiment, food intolerance system 106 may display the action recommendation by sending a text message to client computing device 112 for the user to read. In a further embodiment, food intolerance system 106 may display the action recommendation via food intolerance application 114. In an embodiment, in addition to the action recommendation, food intolerance system 106 may display, for example, food ingredients, food properties, potential food intolerance symptoms, food-related activities to avoid, food-related activities to engage in, timing of food consumption, sequence of food consumption, quantity of food consumption, combinations of food consumption, etc. In an embodiment where client computing device 112 includes a speaker, food intolerance system 106 may speak the action recommendation to the user. In another embodiment, food intolerance system 106 may sound an alarm, such as a beeping noise, to alert the user to a recommendation.

Food intolerance system 106 receives the user action and health condition result (step 318). Food intolerance system 106 receives the user action regarding the food selection, in light of the action recommendation, as discussed with respect to step 214 of FIG. 2. Food intolerance system 106 also receives the health condition of the user, as a result of taking or not taking the action recommendation, as discussed with respect to step 216 of FIG. 2.

Food intolerance system 106 stores the user action and health condition result with corresponding data and action recommendation (step 320). In an embodiment, food intolerance system 106 stores the received food data, the received user data, the received environmental data, the received food-related activity data, as well as the determined current health condition, with the predicted food intolerance symptoms, the recommended action, the user action and the result of the user action. In an embodiment, food intolerance system 106 stored the above described information in knowledge corpus 108.

Food intolerance system 106 feeds the stored data into the food intolerance model (step 322). As part of the learning transfer process of reinforcement learning, food intolerance system 106 feeds any newly acquired data and information relating to the specific user's food intolerance into the food intolerance model such that the food intolerance model continues to learn and increasingly improves the accuracy of food intolerance predictions and action recommendations, thereby personalizing a food intolerance model to the specific user. Learning transfer models have the benefit of often requiring less than ten percent of the data and code to fit a new domain or specialized model.

FIG. 4 depicts a block diagram of components of server computer 104 within distributed data processing environment 100 of FIG. 1, in accordance with an embodiment of the present invention. It should be appreciated that FIG. 4 provides only an illustration of one implementation and does not imply any limitations with regard to the environments in which different embodiments can be implemented. Many modifications to the depicted environment can be made.

Server computer 104 can include processor(s) 404, cache 414, memory 406, persistent storage 408, communications unit 610, input/output (I/O) interface(s) 412 and communications fabric 402. Communications fabric 402 provides communications between cache 414, memory 406, persistent storage 408, communications unit 410, and input/output (I/O) interface(s) 412. Communications fabric 402 can be implemented with any architecture designed for passing data and/or control information between processors (such as microprocessors, communications and network processors, etc.), system memory, peripheral devices, and any other hardware components within a system. For example, communications fabric 402 can be implemented with one or more buses.

Memory 406 and persistent storage 408 are computer readable storage media. In this embodiment, memory 406 includes random access memory (RAM). In general, memory 406 can include any suitable volatile or non-volatile computer readable storage media. Cache 414 is a fast memory that enhances the performance of processor(s) 404 by holding recently accessed data, and data near recently accessed data, from memory 406.

Program instructions and data used to practice embodiments of the present invention, e.g., food intolerance system 106 and database(s) 110, are stored in persistent storage 408 for execution and/or access by one or more of the respective processor(s) 404 of server computer 104 via cache 414. In this embodiment, persistent storage 408 includes a magnetic hard disk drive. Alternatively, or in addition to a magnetic hard disk drive, persistent storage 408 can include a solid-state hard drive, a semiconductor storage device, a read-only memory (ROM), an erasable programmable read-only memory (EPROM), a flash memory, or any other computer readable storage media that is capable of storing program instructions or digital information.

The media used by persistent storage 408 may also be removable. For example, a removable hard drive may be used for persistent storage 408. Other examples include optical and magnetic disks, thumb drives, and smart cards that are inserted into a drive for transfer onto another computer readable storage medium that is also part of persistent storage 408.

Communications unit 410, in these examples, provides for communications with other data processing systems or devices, including resources of client computing device 112 and IoT platform 118. In these examples, communications unit 410 includes one or more network interface cards. Communications unit 410 may provide communications through the use of either or both physical and wireless communications links. Food intolerance system 106, database(s) 110, and other programs and data used for implementation of the present invention, may be downloaded to persistent storage 408 of server computer 104 through communications unit 410.

I/O interface(s) 412 allows for input and output of data with other devices that may be connected to server computer 104. For example, I/O interface(s) 412 may provide a connection to external device(s) 416 such as a keyboard, a keypad, a touch screen, a microphone, a digital camera, and/or some other suitable input device. External device(s) 416 can also include portable computer readable storage media such as, for example, thumb drives, portable optical or magnetic disks, and memory cards. Software and data used to practice embodiments of the present invention, e.g., food intolerance system 106 and database(s) 110, can be stored on such portable computer readable storage media and can be loaded onto persistent storage 408 via I/O interface(s) 412. I/O interface(s) 412 also connect to a display 418.

Display 418 provides a mechanism to display data to a user and may be, for example, a computer monitor. Display 418 can also function as a touch screen, such as a display of a tablet computer.

The programs described herein are identified based upon the application for which they are implemented in a specific embodiment of the invention. However, it should be appreciated that any particular program nomenclature herein is used merely for convenience, and thus the invention should not be limited to use solely in any specific application identified and/or implied by such nomenclature.

The present invention may be a system, a method, and/or a computer program product. The computer program product may include a computer readable storage medium (or media) having computer readable program instructions thereon for causing a processor to carry out aspects of the present invention.

The computer readable storage medium can be any tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, a special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, a segment, or a portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the invention. The terminology used herein was chosen to best explain the principles of the embodiment, the practical application or technical improvement over technologies found in the marketplace, or to enable others of ordinary skill in the art to understand the embodiments disclosed herein.

What is claimed is:

1. A method comprising:
receiving, by one or more computer processors, data, wherein the received data includes data associated with a food item viewed by a first user, data associated with the first user, and data associated with an environment of the first user;
determining, by one or more computer processors, a first health condition of the first user;
predicting, by one or more computer processors, a first food intolerance reaction of the first user to the food item viewed by the first user based on the received data and the determined first health condition;
determining, by one or more computer processors, a first action recommendation for the first user corresponding to the predicted first food intolerance reaction; and
presenting, by one or more computer processors, the first action recommendation to the first user.

2. The method of claim 1, wherein predicting the food intolerance reaction of the first user to the food item viewed by the first user based on the received data and the determined first health condition further comprises:
receiving, by one or more computer processors, data associated with two or more users, wherein the received data includes data associated with a food item viewed by two or more users; data associated with the two or more users, data associated with an environment of the two or more users, and data associated with one or more activities of the two or more users;
determining, by one or more computer processors, a health condition of each of the two or more users;
determining, by one or more computer processors, one or more action recommendations for the two or more users corresponding to a second predicted food intolerance reaction;
receiving, by one or more computer processors, one or more actions of the two or more users;
receiving, by one or more computer processors, one or more health conditions of the two or more users resulting from the one or more actions of the two or more users;
relating, by one or more computer processors, the one or more health conditions of the two or more users resulting from the one or more actions of the two or more users with the received data of the two or more users and the received one or more actions of the two or more users; and
generating, by one or more computer processors, a reinforcement learning food intolerance model for recommending one or more actions associated with food intolerance.

3. The method of claim 2, further comprising, determining, by one or more computer processors, an accuracy of the one or more action recommendations, wherein the accuracy is a number of actions that would have produced desirable results divided by a total number of determined one or more action recommendations.

4. The method of claim 2, further comprising:
receiving, by one or more computer processors, a first action by the first user in response to the first action recommendation;
receiving, by one or more computer processors, a result of the first action associated with a second health condition of the first user;
storing, by one or more computer processors, the first action and the result of the first action with the received data, the second health condition of the first user, and the first action recommendation; and
feeding, by one or more computer processors, the first action and the result of the first action with the received data, the second health condition of the first user, and the first action recommendation into the reinforcement learning food intolerance model.

5. The method of claim 1, wherein the data associated with the food item viewed by the first user is selected from the group consisting of: a type of food, a quantity of food, an ingredient of the food, a property of the food, a frequency of consumption of the food, a quality of the food, a certification associated with the food, a time of food consumption, a sequence of food consumption, and a frequency of food consumption within a threshold duration of time.

6. The method of claim 1, wherein the data associated with the first user is selected from the group consisting of: physical data of the first user, an age of the first user, a height of the first user, a weight of the first user, a medical history of the first user, a food intolerance of the first user, a food allergy of the first user, and a dietary restriction of the first user.

7. The method of claim 1, wherein the data associated with the environment of the first user is selected from the group consisting of: a weather condition, a temperature, a wind speed, a presence of precipitation, a type of precipitation, a sky condition, and a current location.

8. The method of claim 1, wherein the first health condition of the first user is selected from the group consisting of: biometric data, a blood pressure, a heart rate, a respiratory rate, a quantity of calories burned, a quantity of calories consumed, a pulse, an oxygen level, a blood oxygen level, a glucose level, a blood pH level, a salinity of user perspiration, a skin temperature, a galvanic skin response, electrocardiography data, a body temperature, eye tracking data, and mobility data.

9. The method of claim 1, wherein presenting the first action recommendation to the first user further comprises displaying, by one or more computer processors, the first action recommendation in a field of view of the first user in an augmented reality device.

10. The method of claim 1, wherein the received data includes data associated with one or more activities of the first user.

11. A computer program product comprising:
one or more computer readable storage devices and program instructions stored on the one or more computer readable storage devices, the stored program instructions comprising:
program instructions to receive data, wherein the received data includes data associated with a food item viewed by a first user, data associated with the first user, and data associated with an environment of the first user;
program instructions to determine a first health condition of the first user;
program instructions to predict a first food intolerance reaction of the first user to the food item viewed by the first user based on the received data and the determined first health condition;
program instructions to determine a first action recommendation for the first user corresponding to the predicted first food intolerance reaction; and
program instructions to present the first action recommendation to the first user.

12. The computer program product of claim 11, wherein the program instructions to predict the food intolerance reaction of the first user to the food item viewed by the first user based on the received data and the determined first health condition further comprise:
program instructions to receive data associated with two or more users, wherein the received data includes data associated with a food item viewed by two or more users; data associated with the two or more users, data associated with an environment of the two or more users, and data associated with one or more activities of the two or more users;
program instructions to determine a health condition of each of the two or more users;
program instructions to determine one or more action recommendations for the two or more users corresponding to a second predicted food intolerance reaction;
program instructions to receive one or more actions of the two or more users;
program instructions to receive one or more health conditions of the two or more users resulting from the one or more actions of the two or more users;
program instructions to relate the one or more health conditions of the two or more users resulting from the one or more actions of the two or more users with the received data of the two or more users and the received one or more actions of the two or more users; and
program instructions to generate a reinforcement learning food intolerance model for recommending one or more actions associated with food intolerance.

13. The computer program product of claim 12, the stored program instructions further comprising:
program instructions to receive a first action by the first user in response to the first action recommendation;
program instructions to receive a result of the first action associated with a second health condition of the first user;
program instructions to store the first action and the result of the first action with the received data, the second health condition of the first user, and the first action recommendation; and
program instructions to feed the first action and the result of the first action with the received data, the second health condition of the first user, and the first action recommendation into the reinforcement learning food intolerance model.

14. The computer program product of claim 11, wherein the program instructions to present the first action recommendation to the first user further comprise program instructions to display the first action recommendation in a field of view of the first user in an augmented reality device.

15. The computer program product of claim 11, wherein the received data includes data associated with one or more activities of the first user.

16. A computer system comprising:
one or more computer processors;
one or more computer readable storage devices;
program instructions stored on the one or more computer readable storage devices for execution by at least one of the one or more computer processors, the stored program instructions comprising:
program instructions to receive data, wherein the received data includes data associated with a food item viewed by a first user, data associated with the first user, and data associated with an environment of the first user;
program instructions to determine a first health condition of the first user;
program instructions to predict a first food intolerance reaction of the first user to the food item viewed by the first user based on the received data and the determined first health condition;
program instructions to determine a first action recommendation for the first user corresponding to the predicted first food intolerance reaction; and
program instructions to present the first action recommendation to the first user.

17. The computer system of claim 16, wherein the program instructions to predict the food intolerance reaction of the first user to the food item viewed by the first user based on the received data and the determined first health condition further comprise:
program instructions to receive data associated with two or more users, wherein the received data includes data associated with a food item viewed by two or more users; data associated with the two or more users, data associated with an environment of the two or more users, and data associated with one or more activities of the two or more users;
program instructions to determine a health condition of each of the two or more users;
program instructions to determine one or more action recommendations for the two or more users corresponding to a second predicted food intolerance reaction;
program instructions to receive one or more actions of the two or more users;
program instructions to receive one or more health conditions of the two or more users resulting from the one or more actions of the two or more users;
program instructions to relate the one or more health conditions of the two or more users resulting from the one or more actions of the two or more users with the received data of the two or more users and the received one or more actions of the two or more users; and
program instructions to generate a reinforcement learning food intolerance model for recommending one or more actions associated with food intolerance.

18. The computer system of claim 17, the stored program instructions further comprising:
program instructions to receive a first action by the first user in response to the first action recommendation;
program instructions to receive a result of the first action associated with a second health condition of the first user;
program instructions to store the first action and the result of the first action with the received data, the second health condition of the first user, and the first action recommendation; and
program instructions to feed the first action and the result of the first action with the received data, the second health condition of the first user, and the first action recommendation into the reinforcement learning food intolerance model.

19. The computer system of claim 16, wherein the program instructions to present the first action recommendation to the first user further comprise program instructions to display the first action recommendation in a field of view of the first user in an augmented reality device.

20. The computer system of claim 16, wherein the received data includes data associated with one or more activities of the first user.
